(19) United States
(12) Patent Application Publication
(10) Pub. No.: US 2002/0133837 A1
(43) Pub. Date: Sep. 19, 2002

(54) Transgenic animals at a nuclear hormone receptor locus

(76) Inventors: Carrolee Barlow, La Jolla, CA (US); Todd Carter, San Diego, CA (US)

Correspondence Address: KNOBBE MARTENS OLSON & BEAR LLP, 620 NEWPORT CENTER DRIVE, SIXTEENTH FLOOR, NEWPORT BEACH, CA, 92660, US

(21) Appl. No.: 09/810,306

(22) Filed: Mar. 16, 2001

(51) Int. Cl. A01K 67/027

(52) U.S. Cl. 800/18; 800/9

(57) ABSTRACT

The present disclosure relates to an inbred non-human transgenic animal having a multimeric nuclear hormone receptor, comprising a thyroid hormone receptor subunit and a nuclear hormone receptor subunit, wherein either or both the thyroid hormone receptor subunit and a nuclear hormone receptor subunits are mutated such that expression of said multimeric nuclear hormone receptor results in a measurable phenotype. The disclosure also contemplates methods of making inbred non-human transgenic animals. Methods of using the disclosed inbred non-human transgenic animals as models for various diseases and for screening small molecules that are determined to be efficacious in the treatment of such diseases are also disclosed.

### FIELD OF THE INVENTION

The invention described herein relates to the transgenic expression of a nuclear hormone receptor to create a model system for a neurological disease state. One embodiment relates to the generation of a transgenic mouse strain that expresses a mutant thyroid hormone receptor. The homozygous or heterozygous expression of this mutant thyroid hormone receptor results in a transgenic mouse strain that has utility as a model system for studying Attention Deficit Disorder (ADD)/Attention Deficit Hyperactivity Disorder (ADHD).

### BACKGROUND OF THE INVENTION

Nuclear hormone receptor (NHR) proteins form a class of ligand activated receptors that, when bound to specific sequences of DNA, regulate transcription of particular genes or sets of genes within the cell. This NHR class of proteins is thought to control and regulate the development and differentiation of numerous organ systems, behavioral centers in the brain, reproductive systems, and other important bodily functions.

NHR Superfamily

Members of the superfamily of nuclear hormone receptors include transcription factors that are involved in a large variety of morphogenetic processes. The ligand-dependency of the receptor activity allows modulation of activity in vivo during development and opens avenues for research that have medical relevance.

NHR Structural Features

Nuclear hormone receptors have common features that are differentially conserved between the various types of receptors, such as the steroid/thyroid hormone receptor superfamily. Although the functions of the various families differ, each members generally contain a set of conserved structural and functional elements. Steroid/thyroid hormone receptors are ligand-dependent transcription factors that regulate diverse aspects of growth, development, and homeostasis by binding as homodimers or heterodimers to their cognate DNA response elements to modulate transcription of target genes. Typically, a member of the steroid/thyroid hormone receptor superfamily is divided into the following domains: a variable N-terminal transactivation domain, a conserved DNA binding domain (DBD), a variable hinge region, a conserved ligand-binding domain (LBD), and often a C-terminal extension domain. Other domains are also known to exist.

As used herein, the phrase “member(s) of the steroid/thyroid hormone nuclear receptor superfamily” (also known as “intracellular receptors” or “the nuclear receptor superfamily”) refers to hormone binding proteins that operate as ligand-dependent transcription factors, including identified members of the steroid/thyroid hormone nuclear receptor superfamily for which specific ligands have not yet been identified (referred to in the art as “orphan receptors”). Members of the steroid/thyroid hormone nuclear receptor superfamily are characterized by the presence of five domains: N-terminal or activation domain (A/B), DNA binding domain (C), hinge domain (D), ligand binding domain (E), and C-terminal domain (F) (Evans, R. Science 240:889-895, 1988).

Members of the nuclear hormone receptor superfamily include intracellular transcription factors that directly regulate gene expression in response to specific ligand binding. This family of receptors is known to affect a wide variety of biochemical functions, including fatty acid metabolism, reproductive development, detoxification of foreign substances, and other functions.

One family of nuclear hormone receptors is the Retinoid X receptor (RXR) family which is thought to be important in nuclear receptor signaling pathways. Members of the RXR family specifically bind 9-cis-retinoic acid, and analogs thereof, and therefore may be directly involved in the transduction of retinoid signals. In transfected cultured cells and in established cell lines, RXRs can act either as homodimers or heterodimeric partners of a number of other nuclear receptors, including retinoic acid receptors, thyroid hormone receptors, the vitamin D3 receptor, peroxisomal proliferator-activated receptors, Steroid and a number of orphan receptors,

Phenotype Altering Modifications to NHRs

Mutations in various nuclear hormone receptors have been found to result in a variety of disease states. For example, mutations in the androgen receptor (AR) gene impair androgen-dependent male sexual differentiation to various degrees. In addition, mutations in the thyroid hormone receptor are known to result in hypothyroidism and hyperthyroidism, and their attendant phenotypic disease states. In view of these and other disease states associated with nuclear hormone receptor mutations, it is not surprising that this superfamily of proteins is the subject of intensive scientific research.

Attention Deficit Disorder/Attention Deficit Hyperactivity Disorder

It is estimated that from 3 to 10 percent of the population has a condition known as Attention Deficit Disorder (ADD) or Attention Deficit Hyperactivity Disorder (ADHD). The American Psychiatric Association, in the manual entitled Diagnostic and Statistical Manual of Mental Disorders Fourth Edition (DSM IV), lists the criteria of inattention, hyperactivity, and impulsivity, as being indicative of ADHD.

The causes of ADD or ADHD (hereinafter ADD/ADHD) are not well understood. However, symptoms of ADD/ADHD are thought to be caused by neurological dysfunction within the brain. Several studies using PET scans have confirmed that there is a definite difference in brain functioning between a group of individuals diagnosed with ADD/ADHD and those without it. The underlying physiological mechanism that causes ADD/ADHD is still not thoroughly understood and remains under scientific study. One theory holds that ADD/ADHD may result from an imbalance of particular neurotransmitters. Studies have also shown that the areas of the brain associated with attention and concentration showed lower activity in the ADD/ADHD subjects when compared to the control group. Theoretically, these are the very functions that are impaired in persons with ADD/ADHD.

ADD/ADHD may be either inherited or acquired. Recent research in genetics has shown that ADD/ADHD runs in families. The specific chromosomes that are involved have not been fully identified though more clues are being discovered as research progresses.

ADD/ADHD may be acquired through various conditions that cause insult (damage) to the brain. During pregnancy and delivery these include the use of drugs during pregnancy, toxemia, infectious diseases, overexposure to radiation, prematurity, and complicated delivery. After birth these include meningitis, encephalitis, seizures from fever, head injury and lead toxicity. Excessive use of sweets does not cause ADD/ADHD though it may make the symptoms worse for some individuals who already have the condition.

Since many children with ADD/ADHD appear to “outgrow” the condition it can also be looked at as a developmental disorder. Some children seem to develop the ability to pay attention and concentrate later than others do, just as there is a wide range to timing for developing the ability to walk, talk or be potty trained. In the past, children with ADD/ADHD, and other learning disabilities were labeled as “underachievers” in their younger years and then reclassified as “late bloomers” when their development in this area finally caught up with their peers.

Today, many adults look back and realize they had a learning disability, ADD/ADHD as a child that they may or may not have been able to overcome. Some people with ADD/ADHD become very successful. Others, however, continue to have significant symptoms as adults. Some are not able to adapt or compensate on their own and do not receive any help from teachers, parents or professionals. These individuals often drop out of school and may be social dropouts of one kind or another. It is for this last reason that children who exhibit signs of ADD/ADHD or other learning disabilities should receive all the help necessary to spare them the frustration and anguish often associated with these conditions.

Thyroid Hormone Receptor and ADD/ADHD

Thyroid hormone is a molecule that may play a role in the hyperactive and impulsive symptoms of children with ADD/ADHD. At least one study has shown a correlation between elevated levels of certain thyroid hormones and hyperactivity/impulsivity in mice. McDonald, et al., Learn Mem September-October 1998 ;5(4-5):289-301. However, no one has yet discovered a good animal model for analyzing the ADD/ADHD disease state. Thus, it would be advantageous to develop an animal model in order to test compounds for their effect this disease in vivo (Zhang & Lazar, Annu Rev Physiol. (2000) 62:439-66).

### SUMMARY OF THE INVENTION

One embodiment of the invention is an inbred non-human transgenic animal having a dominant-negative mutated nuclear hormone receptor, wherein the transgenic animal has an Attention Deficit Disorder/Attention Deficit Hyperactivity Disorder (ADD/ADHD) phenotype.

Another embodiment of the invention is an inbred non-human transgenic animal having a mutated homodimer or heterodimer partner of a nuclear hormone receptor, wherein the transgenic animal has an Attention Deficit Disorder/Attention Deficit Hyperactivity Disorder (ADD/ADHD) phenotype.

Yet another embodiment of the invention is a method for producing an inbred non-human transgenic animal strain having an Attention Deficit Disorder/Attention Deficit Hyperactivity Disorder (ADD/ADHD) phenotype. The method includes: providing a targeting vector having a dominant negative mutated nuclear hormone receptor polynucleotide sequence; introducing the targeting vector into a plurality of animal embryos to produce a plurality of transgenic animals; and selecting a transgenic animal that exhibits the ADD/ADHD phenotype.

### BRIEF DESCRIPTION OF THE DRAWINGS

FIG. 1 is a diagram showing the targeting of the PV mutation onto the TR-beta gene locus by homologous recombination. (A) Schematic representation of the PV targeting vector. (B) Restriction map of the wild-type TR-beta gene locus. (C) Targeting the PV mutation into the TR-beta gene locus via homologous recombination (TRβPVNeoR). (D). The final targeted locus (TRβPV): The NeoR gene was deleted in vivo by crossing the TRβ^{PV/PV}NeoR mice with prion Cre mice. (E). Genotyping of TRβPVNeoR mice: (a). Diagram: Primers used in the PCR analysis of TRβPVNeoR mice. Panel: PCR analysis of TRβPVNeoR mice. The 757 bp fragment from the TRβPVNeoR mice was detected when the pair of primers M5 and M3 was used in PCR. (b). In a separate PCR using the genomic DNA from the same mice, the wild-type mice yielded a 452 bp fragment (panel) when the pair of primers W5 and W3 was used (Diagram). From the two separate runs of PCR, the genotypes of TRβPVNeoR mice were determined. Lanes 2 and 5: wild-type mice; lanes 3 and 8: TRβPVNeoR mice; lanes 4, 6 and 7: TRβ^{PV/PV}NeoR mice. (F). Genotype analysis of TRβPV mice: Fragments with size of 452 bp and 546 bp obtained by PCR from (a) TRβPV and (b) wild-type mice. (c). The actual genotype analysis.

FIG. 2 is photographs of gels showing RT-PCR results from analysis of total RNA, which was isolated from liver (lanes 1-3) and brown adipocytes (lanes 4-6). RT-PCR was carried out using the primers N and C as described in Methods. (A). Diagram: A 689 bp or 688 bp cDNA fragment was obtained from the mutant RNA and wild-type RNA, respectively. However, only the cDNA from the mutant RNA could be restricted with BamHI to yield two fragments with sizes of 380 bp and 309 bp. (B). Northern blot analysis: The probe was the mouse TRβ1 cDNA. Results were from three mice of each genotype as marked.

FIG. 3 is a bar graph showing the results from the behavioral analysis discussed in Example 3.

FIG. 4 is a bar graph showing the results from the behavioral analysis discussed in Example 4.

### DETAILED DESCRIPTION

Definitions

In the present specification and claims, reference will be made to phrases and terms of art which are defined for use herein as follows:

As used herein, a measurable phenotype means a phenotype that may be measured by an assay.

As used herein, a transgenic organism means an organism comprising exogenous, recombinant polynucleotides in its germ-line such that the exogenous, recombinant polynucleotides are transmitted through the germ-line of the transgenic organism. Plants, animals, particularly mammals other than humans, insects and any other organism whose genetic material may be manipulated by the various techniques of molecular biology, are contemplated for use in embodiments of the described invention.

As used herein, a mutant nuclear hormone receptor is a nuclear hormone receptor protein in which one or more of its constituent amino acids have been mutated. Mutation includes adding or deleting one or more amino acids that are present in the wild-type of the nuclear hormone receptor.

As used herein, a protein having “hormone-binding properties characteristic of a hormone receptor” is any protein that has at least about 10% of the affinity for a hormone receptor as the wild-type ligand or synthetic analog thereof.

As used herein, when it is said that the transcription-activating property of a protein (X) is “characteristic” of that of a hormone receptor (R), it means that, if, when tested in an assay such as the “cis-trans” receptor functionality bioassay system, the rate of expression from a gene (G) (whose transcription is activated by binding of a receptor complexed with hormone or hormone analog) is, when protein (X) is employed in place of receptor (R), at least about 10% that shown when receptor (R) itself is used, as long as, in both the case of the “receptor” (R) and “protein (X)”, the involved cells are bathed in the same or similar concentration of hormone or analog thereof.

As used herein, when it is said that a protein has “hormone-binding or transcription-activating properties characteristic of a hormone receptor”, it is intended that the hormone receptor itself be encompassed within this definition.

As used herein, when it is said that the transcription of a gene (G) is “substantially activated by hormone (H), or hormone analog (aH)”, it means that the transcription of gene (G) is induced by binding of: a hormone/receptor [(H) or (aH)/(R) or (r)] complex to chromatin near where gene (G) is located. Under this definition (R) is meant to designate “wild-type” or unaltered hormone receptors. The lower case (r) notation is meant to designate functional “engineered” or “modified” receptor proteins, or proteins encoded by mRNA variants of “wild-type” receptor genes.

As used herein, the terms “transcriptional control unit”, “transcriptional control element”, “hormone responsive promoter/enhancer element” and “DNA sequences which mediate transcriptional stimulation” mean the same thing, and are used interchangeably.

As used herein, in the phrase “operative hormone responsive promoter/enhancer element functionally linked to an operative reporter gene”, the word “operative” means that the respective DNA sequences (represented by the terms “hormone responsive promoter/enhancer element” and “reporter gene”) are operational, i.e., work for their intended purposes; the word “functionally” means that after the two segments are linked, upon appropriate activation by a hormone-receptor complex, the reporter gene will be expressed as the result of the fact that the “hormone responsive promoter” was activated.

As used herein, the term “receptor-negative” means that no receptor is detectable in the cell, or if it is, only a de minimus amount (i.e., a barely detectable amount) of receptor is present.

As used herein, a “mutant” of a nucleotide sequence means a nucleotide sequence that has been genetically engineered to be different from the “wild-type” or unmodified sequence. Such genetic engineering can include the insertion of new nucleotides into the wild-type sequences, deletion of nucleotides from the wild-type sequences, or a substitution of nucleotides in the wild-type sequences.

A nucleotide sequence having “substantial sequence homology” to another nucleotide sequence refers to a nucleotide sequence with de minimus sequence variations from another nucleotide sequence.

### OVERVIEW

Embodiments of the invention relate to transgenic animals and methods for creating transgenic animals that are models of the ADD/ADHD disease states. These animal models are useful as in vivo tests to screen for compounds that effect ADD/ADHD disease states. For example, the transgenic animals themselves can be used in an assay system wherein various candidate compounds are tested for their effect on the ADD/ADHD phenotype. Alternatively, whole organs, tissue, individual cells or a population of cells in culture can be harvested from the animals and used in an assay to study the effect of candidate compounds on ADD/ADHD disease states.

One embodiment of the invention relates to the discovery that animals having dominant negative mutations of their thyroid hormone receptor gene exhibit a phenotype strongly resembling ADD/ADHD in humans. Thus, creation of dominant negative mutants in other nuclear hormone receptors are anticipated to provide a similar phenotypic result.

Another embodiment of the invention relates to modification of nuclear hormone receptors or their heterodimer/homodimer partner that lead to the ADD/ADHD phenotype. Accordingly embodiments comprise transgenic animals carrying a modified nuclear hormone receptor, or their homodimer or heterodimer partner, that produces the ADD/ADHD phenotype in a transgenic organism. Alterations throughout the structure of a nuclear hormone receptor, or its binding partner such as RXR are anticipated to induce the desired ADD/ADHD phenotype in a transgenic organism. More specifically, dominant negative mutations in the transactivator or ligand binding domain of a NHR that effect the ability of the NHR to interact with receptor partners are contemplated as suitable mutations with which to produce the desired ADD/ADHD phenotype.

Exemplary members of the steroid/thyroid hormone receptor superfamily (including the various isoforms thereof) include steroid receptors (Hager G L, et al., J Steroid Biochem Mol Biol. (2000) 74(5):249-54) such as glucocorticoid receptor (GR), mineralocorticoid receptor (MR), estrogen receptor (ER), progesterone receptor (PR), androgen receptor (AR), vitamin D₃ receptor (VDR), and the like; plus retinoid receptors, such as the various isoforms of retinoic acid receptor (e.g., RARα, RARβ or RARγ, the various isoforms of retinoid X (or 9-cis retinoic acid) receptor (e.g., RXRα, RXRβ, or RXRγ, various isoforms of peroxisome proliferator-activated receptors (e.g., PPARα, PPARγ, PPARδ) and the like; thyroid hormone receptor (T₃R), such as TRα, TRβ, and the like; steroid and xenobiotic receptor (SXR, see for example, Blumberg et al., Genes Dev (1998) 12(20):3195-205), RXR-interacting proteins (RIPs; see, e.g., Seol et al., Mol Endocrinol (1995) 9(1):72-85; Zavacki et al., Proc Natl Acad Sci U.S.A. (1997) 94(15):7909-14) including farnesoid X receptor (FXR; see for example, Forman et al., Cell (1995) 81(5):687-93; Hanley et al., J Clin Invest (1997) 100(3):705-12, O'Brien et al., Carcinogenesis (1996) 17(2):185-90), pregnenolone X receptor (PXR; see for example, Schuetz et al., Mol Pharmacol (1998) 54(6):1113-7), liver X receptor (LXR, see, e.g., Peet et al., Curr Opin Genet Dev (1998) 8(5):571-5), BXR (Blumberg et al., Genes Dev (1998) 12(9):1269-77), insect derived receptors such as the ecdysone receptor (EcR), the ultraspiracle receptor (see, for example, Oro et al., in Nature 347:298-301 (1990)), and the like; as well as other gene products which, by their structure and properties, are considered to be members of the superfamily, as defined hereinabove, including the various isoforms thereof (see, e.g., Laudet, V., J Mol Endocrinol (1997) 19(3):207-26).

Examples of orphan receptors contemplated for use herein include HNF4 (see, for example, Sladek et al., Genes & Development 4:2353-2365 (1990)), the COUP family of receptors (see, for example, Miyajima et al., in Nucleic Acids Research 16:11057-11074 (1988), and Wang et al., Nature 340:163-166 (1989)), COUP-like receptors and COUP homologs, such as those described by Mlodzik et al., Cell 60:211-224 (1990) and Ladias et al., Science 251:561-565 (1991), orphan receptor (OR1; see, e.g., Feltkamp et al., J Biol Chem (1999) 274(15):10421-9), the insect derived knirps and knirps-related receptors, short heterodimer partner (SHP; see, e.g., Seol et al., Mol Cell Biol (1997) 17(12):7126-31), hepatocyte nuclear receptor 4 (HNF4), constitutive androstane receptor (CAR; see, e.g., Forman et al., Nature (1998) 395(6702):612-5), and the like.

In one specific example, transgenic mice were created having dominant negative mutations in the transactivation domain of the thyroid hormone receptor beta gene. The mice were found to have a phenotype corresponding to the ADD/ADHD disease state. Both homozygous and heterozygous mice exhibited greater activity than wild-type mice. However, because the homozygous mice appeared to suffer from a multitude of problems, the heterozygous mice were found to have a greater degree of activity than the homozygous mice. In addition, it was observed that these transgenic mice, unlike the wild-type animals, reduced their level of activity in comparison to wild-type mice upon treatment with methylphenidate, an accepted pharmaceutical agent for the treatment of ADHD/ADD.

It should be realized that these animal models provide a means for testing compounds for their effectiveness on ADD/ADHD. Routes and pharmaceutically acceptable carrier compositions for administering test compounds can be determined by those of ordinary skill in the art. For example, controlled release vehicles are also well-known to those of skill in the pharmaceutical sciences. The technology and products in this art are variably referred to as controlled release, sustained release, prolonged action, depro, repository, delayed action, retarded release and time release; the words “controlled release” as used herein is intended to incorporate each of the foregoing technologies.

Preferably, the test compound is injected, infused or released into the animal at a rate of from about 0.005 to 0.2 mg per minute. The test compound can be administered over a period of time ranging from about 1, 3 or 5 minutes to about 30 minutes, or more.

Administration of each dose of the test compound can occur from once a day to about four times a day, or more. Administration can be subcutaneous, intravenous, intramuscular, intraoccular, oral, transmucosal, or transdermal, and can utilize direct hypodermic or other injection or infusion means, or can be mediated by a controlled release mechanism of the type disclosed above. Any controlled release vehicle or infusion device capable of administering a therapeutically effective amount of the test compound over a period of time ranging from about 1 to about 30 or longer minutes can be used.

Although the work discussed in detail below relates to creating homozygous and heterozygous transgenic animals with mutant thyroid hormone receptor-beta genes, the invention is not so limited. For example, in the embodiment described below, the thyroid hormone receptor gene was mutated to introduce the “PV” mutation into the transactivation domain of the wild-type gene. The PV mutation is a C-insertion at codon 448 of the TRbeta gene and leads to a frame-shift of the carboxyl-terminal 14 amino acids of TRbeta1, resulting in total loss of triiodothyronine (T3) binding and transcriptional activation. As discussed below, this mutant receptor was then introduced into an inbred mouse line and the progeny of that line were analyzed for phenotypic changes. Specifically, the TR-beta receptor having the PV mutation was inserted and expressed in the in-bred mouse line.

It was found that heterozygous and homozygous transgenic mice carrying the mutated thyroid hormone receptor with the PV mutation provided a phenotypic model of the ADD/ADHD disease state. While not intending to be limited by any particular theory, it is believed that this dominant negative mutation introduced into the TR receptor effects the mutant nuclear hormone receptor's ability to interact with other members of the hormone regulated signaling pathways. However, the invention is not intended to be limited only to this particular mutation or mechanism of action.

Modulator Domain

The modulator domain possesses a number of potential sites for mutation that could create phenotypic changes in a transgenic organism expressing the mutant protein. For example, the modulator domain is thought to contain a transcriptional activation function, referred to as AF-1. Studies with estrogen and progesterone receptors have suggested that the modulator domain possesses promoter- and cell context-dependent activities, suggesting that the amino-terminal region of NHRs may interact with cell-specific cofactors. Berry, et al., EMBO J 9:2811-2818 (1990) and Tora, et al., Nature 333:85-188 (1988). It has also been observed that AF-1 activity of both estrogen and progesterone receptors is enhanced through phosphorylation by the mitogen-activated protein kinase (MAPK). Kato, et al., Science 270:1491-1494 (1995). Additionally, various protein kinases have been shown to phosphorylate the amino-terminal domains of specific nuclear receptors. Zhang, et al., Mol Endocrinol 11:823-832 (1997). The modulator domain has also been shown to interact with steroid receptor activators (SRCs). Tremblay, et al., Mol Cell 3:513-519 (1999). Mutations that alter one or more of these functions are expected to alter the in vitro function of the mutated NHR. Thus, the generation of a transgenic organism expressing such a mutant NHR would also likely produce an altered phenotype worthy of additional study. More specifically, inbred-mouse strains expressing a TR-beta receptor having one or more function altering mutations in the modulator domain of the mutant receptor are contemplated as having utility as models for the study of ADD/ADHD and other disease states.

DBD

Nuclear hormone receptors are known to bind DNA as monomers, homodimers, and heterodimers. Interestingly, most of the heterodimeric complexes studied to date contain one of the retinoid X receptors (RXRs) as a common partner. Mangelsdorf and Evan, Cell 83:841-850. Nevertheless, alternative heterodimeric interactions between NHRs have been reported. For example, see Bogazzi, et al., JBC 269:11683-11686 (1994). It is contemplated that mutations in an NHR that alter the ability of a mutant NHR protein to interact normally with its usual dimer partners would effect the phenotype of a transgenic organism expressing such a mutated NHR.

In theory, a mutation in any part of an NHR could alter the ability of the resultant mutant protein to form dimers. Mutations in the DBD of a NHR would be might lead to this result given the structural and functional characteristics of this domain.

The DBD, one of the most conserved domains in a NHR, is composed of two zinc finger motifs and a C-terminal extension (CTE). The DBD has been further divided into subdomains involved in HRE site recognition functions (P-box) and dimerization determinants (D- and DR-boxes). Mutations in any of these subdomains would be expected to alter the functionality of the resulting mutant NHR and therefore likely to produce an altered phenotype in a transgenic organism expressing the mutant NHR sequence. Thus, it is believed that the generation of a transgenic organism expressing such a mutant NHR would also produce an altered phenotype worthy of additional study. More specifically, the generation of inbred-mouse strains expressing a dominant negative nuclear hormone receptor having one or more function altering mutations in the DNA binding domain of the mutant receptor are contemplated as having utility as animal models for the study of ADD/ADHD and other disease states.

Hinge Region

The hinge region, located between the DBD and LBD provides flexibility within the NHR. The flexibility required of some NHRs that allow the DBD to rotate 180° to bind as dimers to both direct and inverted HREs can be considerable. It has also been reported that the hinge region may serve as a binding site of various corepressor proteins. Chen & Evans, Nature 377:454-457 (1995). Mutations in the hinge region that increase or decrease the amount of flexibility in a mutant NHR or mutations that would enhance or decrease interactions between a mutant NHR and a corepressor protein would be expected to result in an altered phenotype when such a mutant NHR was expressed in a transgenic organism.

It is believed that the generation of a transgenic organism expressing such a mutant NHR would also likely produce an altered phenotype worthy of additional study. More specifically, the generation of inbred-mouse strains expressing a TR-beta receptor having one or more function altering mutations in the DNA binding domain of the mutant receptor are contemplated as having utility as animal models for the study of ADD/ADHD and other disease states.

LBD

The LBD is known to participate in ligand binding, dimerization, interaction with heat shock proteins, nuclear localization, and transactivation functions. The LBD can also comprise an activation function (AF-2), which is found at the C-terminal end of the LBD. Mutations in this domain are expected to alter one or more of these delineated functions. Accordingly, recombinant NHRs containing such a mutation would likely produce an altered phenotype when such a mutant NHR was expressed in a transgenic organism. More specifically, the generation of a transgenic organism expressing such a mutant NHR would also likely produce an altered phenotype worthy of additional study. More specifically, it is believed that inbred-mouse strains expressing a TR-beta receptor having one or more function altering mutations in the ligand binding domain of the mutant receptor will have utility as animal models for the study of ADD/ADHD and other disease states.

Cofactors, Corepressors and Receptor Partners

It should be noted that mutations of a NHR may effect how the mutant NHR interacts with other proteins, such as cofactors, co-repressors and receptor partners. Accordingly, mutations in the various proteins that interact with a mutant NHR that result in the ADD/ADHD phenotype can also be targets for mutagenesis and transgenic expression in a transgenic organism. More specifically, mutations in RXR monomers, common partners with TR receptor proteins, as well as mutations in various cofactors, co-repressors, and other molecules that interact with the TR receptor to regulate gene expression may also be targeted for mutation using the teachings disclosed herein (Shibata et al., Recent Prog Horm Res. (1997) 52:141-65.

Preparing An Exogenous Polynucleotide Sequence

Once a recombinant mutant NHR or other protein of interest has been generated containing one or more of the mutations discussed above, the polynucleotide encoding the mutant protein is incorporated into a transgenic vector, or exogenous polynucleotide sequence. This exogenous sequence is introduced into the germ line of an organism to produce a transgenic animal that expresses the mutant protein. Methods of constructing vectors for use in generating transgenic organisms are well known in the art. For example, U.S. Pat. No. 5,817,492, to Saito & Kanegae, entitled “Recombinant DNA viral vector for transfecting animal cells” describes one type of suitable vector. Typically, an exogenous polynucleotide sequence is prepared in a vector containing an origin of replication, a promoter, recombination recognition sites, a poly(A) sequence, a drug selection marker, and a foreign gene of interest. In this case, the foreign gene of interest is typically a recombinant nuclear hormone receptor.

Methods Of Generating Transgenic Animals

The invention further provides for transgenic animals, which can be used for a variety of purposes, e.g., to identify ADD/ADHD therapeutics. Methods for obtaining transgenic and knock-out/knock-in non-human animals are well known in the art. For example, the general steps utilized to produce a transgenic animal include preparing an exogenous polynucleotide sequence that encodes a recombinant nuclear hormone receptor, introducing the polynucleotides sequence into an organism such that a transgenic organism is produced, and analyzing the resultant phenotype produced in the transgenic animal by the introduction of the polynucleotide sequence.

An exogenous polynucleotide sequence may also be introduced into an organism using a variety of recombinant DNA methods well known in the art. The method used to introduce the exogenous polynucleotide into the organism will influence the construction of the polynucleotide. Accordingly, a brief discussion of transgenic methodology is appropriate before discussing the construction of the exogenous polynucleotide sequence.

There are presently several general types of techniques available with which to create transgenic animals, including microinjection, retrovirus-mediated transgenesis, and embryonic stem cell mediated transfer. The first transgenic animals were created using DNA microinjection and is described herein. (Proc. Natl. Acad. Sci. U.S.A.. December 1980;77(12):7380-4). Two other techniques, retrovirus-mediated transgenesis (Proc. Natl. Acad. Sci. U.S.A. April 1976;73(4):1260-4) and embryonic stem (ES) cell-mediated gene transfer (Proc. Natl. Acad. Sci. U.S.A. December 1986;83(23):9065-9) are also contemplated.

DNA Microinjection

This method involves the direct microinjection of exogenous nucleic acid into the pronucleus of a fertilized ovum. The introduced nucleic acid leads to altered patterns of gene expression in the transgenic organism. The insertion of DNA is a random process and thus there is a high probability that the introduced gene will not insert itself into a site on the host DNA that will permit its expression. The manipulated fertilized ovum is transferred into the oviduct of a recipient female, or foster mother that has been induced to act as a recipient by mating with a vasectomized male. A major advantage of this method is its applicability to a wide variety of species.

Embryonic Stem Cell-Mediated Gene Transfer

This method involves prior insertion of the desired DNA sequence by homologous recombination into an in vitro culture of embryonic stem (ES) cells. Stem cells are undifferentiated cells that have the potential to differentiate into any type of cell (somatic and germ cells) and therefore to give rise to a complete organism. These cells are then incorporated into an embryo at the blastocyst stage of development. The result is a chimeric animal. ES cell-mediated gene transfer is the method of choice for gene inactivation, the so-called knock-out method.

This technique is of particular importance for the study of the genetic control of developmental processes. This technique works particularly well in mice. It has the advantage of allowing precise targeting of defined mutations in the gene via homologous recombination.

Retrovirus-Mediated Gene Transfer

To increase the probability of expression, gene transfer in this embodiment is mediated by means of a carrier or vector, generally a virus or a plasmid. Retroviruses are commonly used as vectors to transfer genetic material into the cell, taking advantage of their ability to infect host cells in this way. Offspring derived from this method are chimeric, i.e., not all cells carry the retrovirus. Transmission of the transgene is possible only if the retrovirus integrates into some of the germ cells.

For any of these techniques the success rate in terms of live birth of animals containing the transgene is extremely low. Providing that the genetic manipulation does not lead to abortion, the result is a first generation (F1) of animals that need to be tested for the expression of the transgene. Depending on the technique used, the F1 generation may result in chimeras. When the transgene has integrated into the germ cells, the so-called germ line chimeras are then inbred for 10 to 20 generations until homozygous transgenic animals are obtained and the transgene is present in every cell. At this stage embryos carrying the transgene can be frozen and stored for subsequent implantation.

Genetic Techniques

Genetic techniques, which allow for the expression of transgenes can be regulated via site-specific genetic manipulation in vivo, are known to those skilled in the art. For instance, genetic systems are available which allow for the regulated expression of a recombinase that catalyzes the genetic recombination of a target sequence. As used herein, the phrase “target sequence” refers to a nucleotide sequence that is genetically recombined by a recombinase. The target sequence is flanked by recombinase recognition sequences and is generally either excised or inverted in cells expressing recombinase activity. Recombinase catalyzed recombination events can be designed such that recombination of the target sequence results in either the activation or repression of expression of one of the subject proteins, or increase or decrease in the activity of the subject protein. In an illustrative embodiment, either the cre/loxP recombinase system of bacteriophage P1 (Lakso et al. (1992) PNAS 89:6232-6236; Pichel et al. (1993) Oncogene 8: 3333-42; Orban et al. (1992) PNAS 89:6861-6865) or the FLP recombinase system of Saccharomyces cerevisiae (O'Gorman et al. (1991) Science 251:1351-1355; PCT publication WO 92/15694) can be used to generate in vivo site-specific genetic recombination systems. Cre is a 38 kDa recombinase protein from bacteriophage P1 that mediates intramolecular (excisive or inversional) and intermolecular (integrative) site specific recombination between loxP sites (reviewed in Methods of Enzymology; 1993, Vol. 225, 890-900). A loxP site consists of two 13 bp inverted repeats separated by an 8 bp asymmetric spacer region to which the Cre recombinase binds and are required for Cre recombinase mediated genetic recombination. The orientation of loxP sequences determines whether the intervening target sequence is excised or inverted when Cre recombinase is present (Abremski et al. (1984) J. Biol. Chem. 259:1509-1514); catalyzing the excision of the target sequence when the loxP sequences are oriented as direct repeats and catalyzes inversion of the target sequence when loxP sequences are oriented as inverted repeats.

Any technique which allows for the addition of the exogenous genetic material into nucleic genetic material can be utilized so long as it is not destructive to the cell, nuclear membrane or other existing cellular or genetic structures. The exogenous genetic material is preferentially inserted into the nucleic genetic material by microinjection. Microinjection of cells and cellular structures is known and is used in the art.

Reimplantation is accomplished using standard methods. Usually, the surrogate host is anesthetized, and the embryos are inserted into the oviduct. The number of embryos implanted into a particular host will vary by species, but will usually be comparable to the number of off spring the species naturally produces.

Transgenic offspring of the surrogate host may be screened for the presence and/or expression of the transgene by any suitable method. Screening is often accomplished by Southern blot or Northern blot analysis, using a probe that is complementary to at least a portion of the transgene. Western blot analysis using an antibody against the protein encoded by the transgene may be employed as an alternative or additional method for screening for the presence of the transgene product. Typically, DNA is prepared from tail tissue and analyzed by Southern analysis or PCR for the transgene. Alternatively, the tissues or cells believed to express the transgene at the highest levels are tested for the presence and expression of the transgene using Southern analysis or PCR, although any tissues or cell types may be used for this analysis.

Alternative or additional methods for evaluating the presence of the transgene include, without limitation, suitable biochemical assays such as enzyme and/or immunological assays, histological stains for particular marker or enzyme activities, flow cytometric analysis, and the like. Analysis of the blood may also be useful to detect the presence of the transgene product in the blood, as well as to evaluate the effect of the transgene on the levels of various types of blood cells and other blood constituents.

When evaluating for the presence of the transgene, often a selectable marker such as a reporter or a drug selectable marker, e.g., neomycin resistance (NeoR) gene, is typically inserted into an intron near the desired alteration. Unfortunately, in many cases the presence of the drug resistance gene interferes with insertion at the normal locus resulting in a null or hypomorphic allele (Nagy et al. (1998) Curr Biol, 8(11), 661-4; Xu et al. (1999) Nat Genet, 22(1), 37-43). To overcome this problem, the drug resistance gene, flanked by loxP sites, can be removed by exposure to Cre recombinase (Cre). Cre can be introduced by several methods. First, a Cre-expressing plasmid can be introduced into ES cells (Xu et al. (1999) Nat Genet, 22(1), 37-43). Also, a Cre-expressing plasmid can be introduced into fertilized eggs (Araki et al. (1995) Proc Natl Acad Sci U.S.A. , 92(1), 160-4., Sunaga et al. (1997) Mol Reprod Dev, 46(2), 109-13). Finally, a mouse mutant created using ES cell technology can be mated to a transgenic mouse that expresses Cre in the germ line (Williams-Simons, L. and Westphal, H. (1999) Transgenic Res, 8(4), 53-4). However, most transgenic mice are produced in a mixed background so that subsequent mice are outbred. This can be problematic for phenotypic analysis in many organ systems, including the brain, and for the analysis of behavior (Williams-Simons, L. and Westphal, H. (1999) Transgenic Res, 8(4), 53-4, Sandberg et al.. (in press) PNAS; and Crusio, W. E. (1996) Trends Neurosci, 19(5), 186-7; discussion 188-).

In order to maintain an inbred line, transfection into ES cells or pronuclear injection of a Cre-expressing plasmid is preferably used. Unfortunately, both of these methods are labor intensive. In order to overcome this problem, one method employed in the present example was to generate Cre-expressing mice in a 129SvEv background that show germ line deletion of a loxP-flanked NeoR allele (loxP-NeoR). This reagent can now be used to delete loxP-flanked sequences in all tissues while maintaining a background isogenic to that of the commonly used 129SvEv ES cells.

Progeny of the transgenic animals may be obtained by mating the transgenic animal with a suitable partner, or by in vitro fertilization of eggs and/or sperm obtained from the transgenic animal. Where mating with a partner is to be performed, the partner may or may not be transgenic and/or a knockout; where it is transgenic, it may contain the same or a different transgene, or both. Alternatively, the partner may be a parental line. Where in vitro fertilization is used, the fertilized embryo may be implanted into a surrogate host or incubated in vitro, or both. Using either method, the progeny may be evaluated for the presence of the transgene using methods described above, or other appropriate methods.

Transgenic Organisms as Disease Model Systems

The transgenic organisms generated by the methods described herein have utility as model systems to test for new drug compounds that would affect ADD/ADHD. For example, test compounds can be given in various doses to the transgenic animals. A dose/response curve can then be generated in order to study the effect of the test compound on ADD/ADHD. Compounds that are found to reduce the activity of the animals can then be chosen for further study.

As discussed below, the utility of the model system described herein has been demonstrated by observations that transgenic mice with mutated thyroid hormone receptor genes displayed increased physical activity as compared to a wild type control. Additionally, we found that the level of activity in these mice was reduced in comparison to wild-type mice upon treatment with the pharmaceutical compound methylphenidate which is an efficacious treatment for ADD/ADHD.

Tissue Based, Cellular and Sub-cellular Assay Systems

In addition to using transgenic animals as a model system for ADD/ADHD, there is great benefit to looking at the brains of these animals, in whole or in part, as assay systems with which to screen compounds that effect ADD/ADD. In one embodiment, brain tissue harvested from a transgenic animal generated according to the methods taught herein are assayed for changes in various measures of brain metabolism and activity, when the tissue is exposed to test compounds with potential activity in treating ADD/ADHD.

For example, gene expression analysis, glucose metabolism rates, neurotransmitter turnover, neuronal activity, ATP hydrolysis, and other metabolic indicators can be monitored as tests with which to assay potential ADD/ADHD drugs using standard methods well known in the art. These assays can be performed using whole brain or utilizing specific regions of the brain thought to be involved in the ADD/ADHD phenotype.

One example of a gene expression analysis is discussed in Sandberg, et al., Proc. Natl. Acad. Sci. U.S.A. Sep. 26, 2000;97(20):11038-43. In this study, differences in gene expression were analyzed using different brain regions of two mouse strains. The expression patterns from each mouse strain were examined in response to a drug that causes seizure. The Sandberg, et al. paper was directed to studying the hippocampus for expression of seizure-responsive genes. Nevertheless, a similar methodology can be used to test candidate compounds for activity against ADD/ADHD, or other neurological or nuclear hormone-related diseases of interest.

Methods of measuring changes in neurotransmitter level in response to the administration are well known in the art. An exemplary method of measuring neurotransmitter levels in response to a test compound is found drug, is found in Ito, R., et al., “Dissociation in conditioned dopamine release in the nucleus accumbens core and shell in response to cocaine cues and during cocaine-seeking behavior in rats,” J Neurosci. Oct. 1, 2000;20(19):7489-95. It should be noted that there are many other methods for measuring neurotransmitter levels that would have utility in the present invention.

In terms of measuring neuronal activity, the work of Chang, et al., Neuroscience. 2000;99(3):433-43, provides a teaching that can be adapted to test a library of compounds of interest for those molecules that have activity against ADD/ADHD or other phenotypes induced by the expression of a mutant nuclear hormone receptor in a transgenic tissue or cell.

Dopamine release is measured by preparing synaptosomes from the striatal area of a transgenic animal, such as a transgenic mouse. Methods of making synaptosomes, are generally known in the art and are exemplified by the procedures set forth by Nagy et al., J. Neurochem., Vol. 43, pp. 1114-1123 (1984).

Ganglionic effects of compound of interest can be assayed using a variety of methods well known in the art. In one embodiment, immortalized neural cells can be used to assay populations of candidate compounds for utility in treating ADD/ADHD. Ganglionic effects of other compounds have been tested in the rat pheochromocytoma clonal line PC12, which is a continuous clonal cell line of neural crest origin derived from a tumor of the rat adrenal medulla expressing ganglionic-type neuronal nicotinic receptors (see Whiting et al., Nature, Vol. 327, pp. 515-518 (1987); Lukas, J. Pharmacol. Exp. Ther., Vol. 251, pp. 175-182 (1989); Whiting et al., Mol. Brain Res., Vol. 10, pp. 61-70 (1990)). See also, U.S. Pat. No. 5,731,314.

### EXAMPLES

Without further elaboration, it is believed that one skilled in the art can, using the preceding description, utilize the present invention to its fillest extent. The following examples are illustrative only, and not limiting of the remainder of the disclosure in any way whatsoever.

### Example 1

Generation of Inbred Transgenic Mice with a Targeted Mutation in the Thyroid Hormone β Receptor Gene

To understand the molecular basis underlying the action of mutant TRβ in vivo, transgenic mice were generated with a targeted mutation in the TRβ gene (TRβPV) using homologous recombination and the Cre/loxP system.

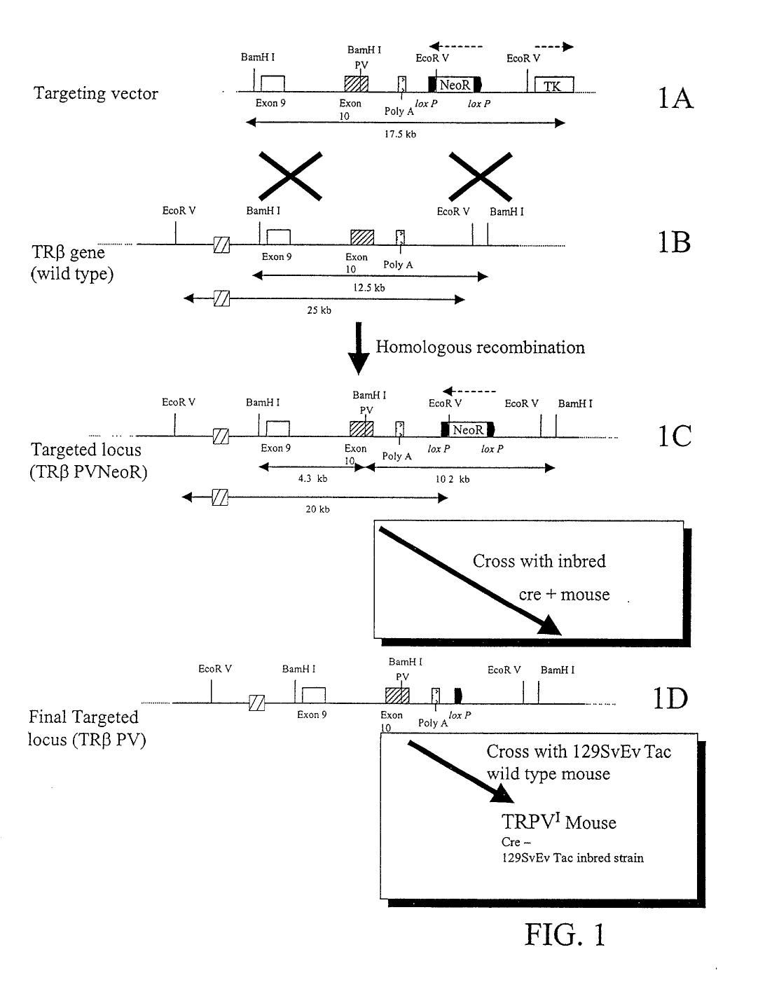

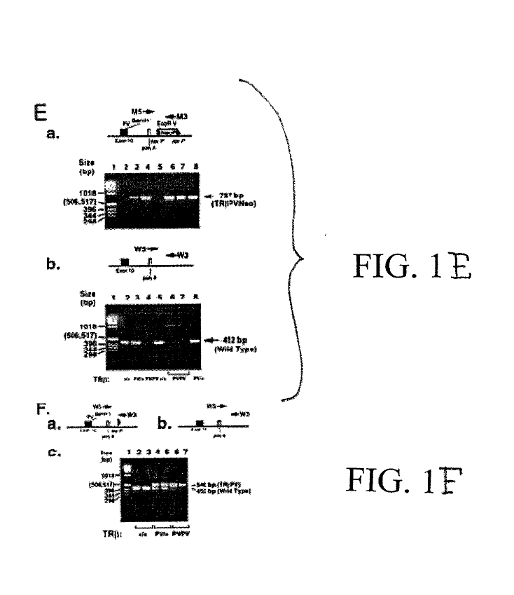

A TRβ mouse genomic clone was isolated from an ES-129 P1 Library (GenomeSystems, Inc., St. Louis, Mo.) using primers specific for the TRβ gene. After mapping the genomic sequences in the regions of exons 8, 9, 10 and the down-stream sequences of exon 10, a 17.5-kb targeting vector was constructed (see FIG. 1A). A PV mutation site was generated in exon 10 with a C insertion at the coding nucleotide position 1642. In addition, a BamH I site was introduced at the coding nucleotides 1668-1673 without changing the amino acid sequence of the PV mutant. The neomycin resistance gene (NeoR), flanked by two LoxP sequences, was placed 1 kb downstream of the polyA site. The thymidine kinase gene (TK) was placed at the 3′-terminus of the targeting vector. The length of the 5′ and 3′ arms were 5.4 kb and 5.6 kb, respectively.

The targeting vector was linearized by XbaI digestion and transfected (25 μg) into the TC-1 ES cells (Barlow et al. (1996) Cell 86, 159-171). Recombinant clones were treated with 400 μg/ml G418 for positive selection and 2 μM gancyclovir for negative selection. Seven recombinant clones were identified by southern blotting analysis. The clones were microinjected into C57BL/6J blastocysts to produce chimeras that were crossed with NIH Black Swiss mice to establish germ line transmission (Deng et al. (1994) Genes & Dev. 8, 3045-3057). Mice harboring the targeted PV mutation and the NeoR gene are designated as TRβPVNeoR mice (FIG. 1C).

TRβPVNeoR mice were genotyped by Southern blotting analyses and/or by PCR using two sets of primers. For the identification of the wild-type allele, the sequence of the 5′-primer (W5) is: (5′-TCCCCAAGCCAGCATCCCGACC)(SEQ ID NO: 1) and the 3′-primer (W3) is: (5′-CGGGCAAATCCTTACCTGG)(SEQ ID NO: 2). For the identification of the mutant allele, the 5′-mutant primer (M5) is: (5′-TGCAGTGGCGATGGCATC)(SEQ ID NO: 3) and 3′-primer (M3) is: (5′-CTGACCGCTTCCTCGTGCTTTACG)(SEQ ID NO: 4). PCR reactions were carried out for 35 cycles (94° C., 30 sec; 59° C., 30 sec; 74° C.; 30 sec; for the determination of the wild-type allele) (94° C., 30 sec; 63° C., 30 sec; 74° C; 30 sec. for the mutant-allele) in a buffer containing MgCl₂ using TaKaRa EX-Taq polymerase (INTERGEN, New York, USA).

To delete the NeoR gene from the targeted locus in vivo, TRβPVNeoR mice (FIG. 1C) were crossed with homozygous prion-Cre transgenic mice (Lakso et al. (1996) Proc. Natl. Acad. Sci. U.S.A. 93, 5860-5865). The resulting offspring were genotyped by Southern blot and PCR analyses using primers W5 and W3. Mice harboring the targeted PV mutation, but without the NeoR gene, are designated as TRβ^{PV/PV} and TRβ^{PV/+} for the homozygous and heterozygous mutant mice, respectively.

Determination of the Expression of PV Mutant RNA in Tissues by RT-PCR

Total RNA was prepared using Trizol® Reagent (Life Technologies, Gaithersburg, Md.). Reverse transcription-polymerase chain reaction (RT-PCR) was carried out using poly-d(T) as a primer for cDNA synthesis using SuperScript™ II reverse transcriptase (Life Technologies, Gaithersburg, Md.). Fragments with the size of 688 bp from the wild-type TR or of 689 bp from the mutant PV were amplified in the presence of 5′-primer (primer N):

(5′ ATGGGGAAATGGCAGTGACACGAG) (SEQ ID NO: 5) and 3′-primer (primer C):

(5′-TGGGAGCTGGTGATGACTTCGTGC) (SEQ ID NO: 6) using Taq DNA polymerase (Perkin-Elmer, Branchburg, N.J.). The mutant PV sequence contained a BamH I site which was not present in the mouse endogenous TRβ gene. PCR products were digested with BamH I to yield two fragments with the size of 380- and 309-bp for mutant PV as analyzed by gel electrophoresis.

Northern Blot Analyses

Total RNA (5-10 μg) was used for northern blot analysis. After electrophoresis, RNA was transferred onto membranes (Hybond-N+, Amersham Life Science, Arlington Height, Ill.) which were hybridized with appropriate probes. cDNA clones for TSHα, TSHβ, growth hormone (GH), malic enzyme (ME), spot 14 and 5′-deiodinase I were labeled with [α-³²P]dCTP using a random primer hexamer protocol. For quantification, the intensities of the mRNA bands were normalized against the intensities of GAPDH mRNA (n=3). The blots were stripped and re-hybridized with a ³²P-labeled GAPDH cDNA. Quantification was performed using the Molecular Dynamics PhosphorImager (Molecular Dynamics, Amersham Pharmacia Biotech, Inc., Piscataway, N.J.).

Hormone Assays and Determination of Serum Cholesterol

Serum levels of total T4 and T3 were determined using Gamma Coat T4 or T3 assay RIA kit (DiaSolin Inc, Stillwater, Minn.) according to the manufacture's instruction. TSH levels in serum were measured as described (Pohlena et al. (1999) Thyroid 9, 1265-1271).

Histology and Immunohistochemistry

Thyroid glands isolated from the transgenic mice were fixed in 10% neutral buffered formalin and subsequently embedded in paraffin. Five μm-thick sections were prepared and stained with hematoxylin and eosin for microscopic examination.

Pituitaries from TRβ^{PV/PV} mice and wild-type litter mates were fixed in 10% formalin in phosphate-buffered saline (PBS) for 2 hours at 23° C., dehydrated and embedded in paraffin. Five μm-thick sections were deparaffinized in xylene, washed in ethanol, then treated with antigen unmasking solution (Vector Labs, Burlingame, Calif.) and heated in a microwave oven for 5 minutes. The sections were then blocked in 1% bovine serum albumin in PBS for 30 minutes at 23° C., followed by sequential incubations in rabbit anti-mouse TSH antiserum (1:1000 dilution in bovine serum albumin-PBS; Biogenesis [Poole, UK; cat.#8922-6009)] for 30 minutes at 23° C., washing with PBS, then affinity-purified goat anti-rabbit IgG conjugated to horseradish peroxidase (Jackson ImmunoResearch, West Grove, Pa.). Following washing in PBS, the sections were incubated in diaminobenzidine substrate solution (10 min, 23° C.), followed by dehydration and mounting under coverslips without counterstains. Unstained sections were then examined using bright field microscopy, as well as phase contrast microscopy, and digital images were captured from these sections using a 20×objective and a Dage 300 CCD camera with a Zeiss Axioplan 2 microsope. These images were then printed, and nuclei of all cells in the field were counted using the phase contrast image. The cells showing TSH labeling were then counted in the parallel bright field image of the same field. The total number of cells counted for the pituitaries from the TRβ^{PV/PV} mice and wild-type mice were 866 and 655, respectively.

Data Analysis

All data are expressed as mean±S.E. Statistical analyses utilized the student's t test and p<0.05 was considered significant.

### Results

Generation of Mice with Targeted TRβ Mutant PV

The targeting vector is shown in FIG. 1A. Positive ES clones were identified using both internal and external probes (FIGS. 1B and C). BamHI digests of the genomic DNA from two positive clones revealed 12.5-, 10.2- and 4.3-kb fragments when hybridized with the internal probe encompassing the PV mutation site in exon 10 ; whereas, the control showed the expected 12.5-kb band. When the EcoRV digests of the same positive ES clones were hybridized with the external probe, an additional 20-Kb fragment was detected together with the 25-kb fragment derived from the wild-type allele. These results indicate that the PV mutation was correctly targeted onto the TRβ gene locus.

Mice carrying the TRβPVNeoR gene (FIG. 1C) were identified by PCR using DNA extracted from tails. Using primers for detection of mutant genes (M5 and M3; top panel of FIG. 1E-a) or wild-type (W5 and W3; top panel of FIG. 1E-b), typical results are shown in the lower panels of FIGS. 1E-a and b, respectively. The size of PCR products were 757 bp (lanes 3, 4, 6, 7 and 8 from different mice; lower panel of FIG. 1E-a) and 452 bp (lanes 2, 3, 5 and 8; lower panels of FIG. 1E-b) from the mutant and wild-type genes, respectively.

Northern blot analyses of PV gene expression in TRβ^{PV/+}NeoR heterozygous and TRβ^{PV/PV}NeoR homozygous mice (FIG. 1C) indicate that the expression of the PV mutant allele was repressed as compared to the wild-type TRβ allele . Because the NeoR gene was flanked by two loxP sequences in the targeted locus (FIGS. 1A and C), it was possible to delete the NeoR gene in vivo by crossing the TRβ^{PV/PV}NeoR homozygous mice with an prion-Cre mice (Lakso et al. (1996) Proc. Natl. Acad. Sci. U.S.A. 93, 5860-5865). PCR analysis using primers W5 and W3 (FIG. 1F) yielded a 546 bp fragment indicative of the removal of the NeoR gene (see FIGS. 1B, C and D). Mice with the final targeted locus (without NeoR gene: FIG. 1D) were designated as TRβ^{PV/+} for the heterozygous mice and TRβ^{PV/PV} for the homozygous PV mutant mice.

The PV Gene was Expressed in all Expected Target Tissues Examined

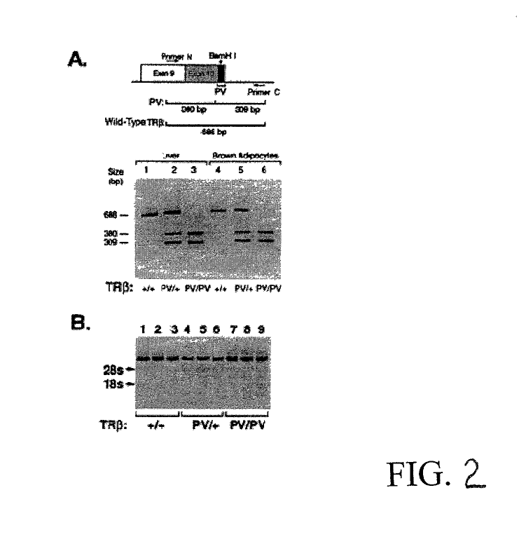

RT/PCR was used to assess the expression of the PV mutant allele at the RNA level. Primers flanking the mutated exon 10 were used and the resultant cDNA was digested with BamH I (top panel of FIG. 2A). The cDNA derived from the mutant allele yielded two fragments with sizes 380- and 309-bp; whereas, a 688-bp fragment was obtained from the wild-type mRNA. FIG. 2A (lower panel) shows the representative mRNA expression patterns in the liver and brown adipocytes of TRβ^{PV/+} mice (lanes 2 and 5) and TRβ^{PV/PV} mice (lanes 3 and 6), respectively. This analysis shows that PV allele was expressed in the cerebrum, cerebellum, pituitary, heart, muscle, white adipocytes, lung, spleen and kidney, indicating that the PV mutant allele was detected in expected T3 target tissues.

The expression of the PV allele in the liver was further confirmed by northern blot analysis using a cDNA probe specific to the TRβ gene (FIG. 2B). Lanes 7-9 (from three mice) indicate that the level of the transcription of TRβPV gene in the TRβ^{PV/PV} mice was not significantly different from those in the TRβ^{PV/+} (lanes 4-6 for three mice) and in the wild-type TRβ^{+/+} litter mates (lanes 1-3 for three mice). Similarly, expression of the PV and wild-type TRβ allele were similar in the heart and brain. These findings indicate that the expression of the TRβPV gene was not affected by the presence of the PV mutation.

### Example 2

Generation of an Inbred 129SvEV GFPCre Transgenic Mouse for use in Deleting loxP-flanked Genes.

A common method for generating tissue specific mutations in mice relies on using transgenic mice expressing Cre recombinase to delete genes flanked by loxP sites. Often however, the presence of a drug resistance gene interferes with the normal locus and creates a null or hypomorphic allele. Removal of a selectable marker flanked by loxP sites can overcome this problem. The most simple method used to remove a loxP-flanked selectable marker is to breed an animal carrying a loxP-flanked allele to a strain that expresses Cre recombinase in the germ line. In order to maintain a mutation on an inbred background, however, the transgenic Cre-expressing mouse must be isogenic to that of the embryonic stem (ES) cells used. A transgenic 129SvEv inbred strain was generated that expresses Cre recombinase and allows excision of loxP-flanked sequences in all tissues. This reagent allows deletion of loxP-flanked sequences while maintaining the mutation on an inbred background.

Transgene Construct

The polynucleoide sequence for green fluorescent protein (GFP) was removed from the vector pEGFP-C1 (Clontech, Palo Alto, Calif.) using Eco47111/Xho1. The GFP sequence was isolated by gel purification and inserted into EcoRV/Xho1 digested pBSKS (Strategene, San Diego, Calif.) to generate pBSKSGFP. The 1.1 kb Cre recombinase gene was amplified using the plasmid pCre306C as a template (courtesy of Steve O'Gorman) using the 5′ primer 5′-GCT AGC TCC GGA GAG CAA AAGCTG-3′ (SEQ ID NO: 7) and the 3′ primer 5′-CTC TAG CTC GAG CTA ATC GCC ATC-3′ (SEQ ID NO: 8) which included a 5′-Bspe1 site and a 3′ Xho1 site for use in generating an in frame fusion of Cre to GFP. The product was gel purified and the fragment inserted in pBSKSGFP at the Bspe 1/Xho1, to generate pBSKSGFP/Cre. The entire GFPCre fusion product was sequenced verified.

The final transgenic vector was constructed by inserting the purified 1.86 kilobase blunted Not1 GFPCre fusion cDNA into blunted Kpn1 and Not1 sites in the plasmid hgPrnp (Weissmann et al. (1996) Cold Spring Harb Symp Quant Biol, 61, 511-22 and Fischer et al. (1996) Embo J, 15(6), 1255-64) downstream of a modified prion promoter. The Prion.GFPCre plasmid was purified using CsCl ultracentrifugation×2 according to published methods (Hogan et al. (1994) Manipulating the mouse embryo: a laboratory manual, Second Edition Ed., Cold Spring Harbor Laboratory Press, Cold Spring Harbor, N.Y.). The purified plasmid was digested using Mlu1 and Sal1 to release the backbone. The 5.7 kilobase Prion.GFPCre fragment was isolated after separation on a 1% agarose gel overnight at 4° C. using Gene Clean (Bio 101, CA).

Production of Transgenic 129SvEv Mice

All animal procedures were performed according to protocols approved by both The Salk Institute for Biological Studies and the National Institutes of Health animal care and use committees. The procedures for superovulation, embryo recovery, microinjection, and transfer were similar to those described Hogan, 1994. Female 129SvEv mice at 4 weeks of age were used as embryo donors after superovulation with 2.5 units of PMS or gonadotropin—pregnant mare's serum (Calbiochem, cat.#367222) and 2.5 units of chorionic gonadotropin (Organon, cat.#0052-0315-10) 46 hrs apart. Stud males were 129SvEv. Recipient females used were CBy6F1 (8 weeks or older) and were placed with CB6F1 vasectomized males. The microinjections were performed at 200×magnification using a Zeiss Axiovert 135M, Eppendorf micromanipulator 5171, and the automatic Eppendorf microinjector 5242. Embryos were injected in M2 (Speciality Media cat. #MR-015-D), and then incubated in BMOC2 (Speciality Media cat#MR-013-D) at 37° C. in 5% CO2 overnight. The 2-cells were separated from the 1-cells the following morning and surgically transferred to primed recipients.

Analysis of Offspring

Genotypes of all offspring were analyzed by DNA polymerase chain reaction (PCR) using DNA prepared from tail biopsies. DNA was prepared using standard protocols (Sambrook, J., Fritsch, E. F. and Maniatis, T. (1989) Molecular Cloning: A Laboratory Manual, Second Edition Ed., Cold Spring Harbor Laboratory Press, Cold Spring Harbor, N.Y.). Cre positive mice were identified using a 5′ Cre specific primer 5′-CCGGGCTGCCACGACCAA-3′ (SEQ ID NO: 9) and 3′ Cre specific primer 5′-GGCGCGGCAACACCATTTTT-3′ (SEQ ID NO: 10). To identify deletion of a loxP-flanked gene, DNA was prepared from several brain regions, kidney, testes and liver using Phase-Lock Gel reagent (5 Prime-3 Prime, Inc., Boulder, Colo.) after overnight lysis of the tissue. PCR was performed on the DNA using primers which amplified either a wild type allele, a loxP-flanked NeoR allele or an allele where the loxP-flanked NeoR gene was deleted leaving a single intact loxP site. PCR reactions were run on 0.8% agarose TBE gels.

Immunoflourescence

Mice were perfused with cold 4% parafomaldehyde transcardially. The kidney, testes, brain, and liver were dissected and submerged in 30% sucrose for two days before microtome sectioning (40 μm). Sections were stained with DAPI, analyzed and photographed using confocal microscopy (BioRad, Hercules, Calif.).

Results: Production of Transgenic 129SvEv Mice

Of 40 129SvEv superovulated females, approximately 200-250 embryos were harvested and 97 embryos at the 2-cell stage were recovered after microinjection. These embryos were implanted and 11 pups were born. Screening by PCR identified two Cre positive founder animals. Both founders were bred to 129SvEv wild type mice to generate inbred F1 offspring. Only founder 98030B produced Cre positive transgenic offspring.

Cre Mediated Deletion of a loxP-flanked NeoR Gene

Tail DNA was isolated and PCR amplification was used to determine if Cre-expressing mice showed deletion of the loxP-flanked NeoR gene. To generate the appropriate mice a female loxP-NeoR heterozygous mouse was bred to the Prion.GFPCre transgenic founder and a resulting Cre positive male offspring was studied. The 187 base pair wild type allele was amplified in both the breeder female and the male offspring. However, an additional 221 base pair fragment was amplified in the Cre-expressing offspring, suggesting that there was excision of the NeoR gene and retention of a single loxP site. To determine the extent of Cre-mediated excision, PCR was used to amplify the intact NeoR allele. The breeder female had an intact loxP-NeoR allele whereas the Cre-expressing male offspring showed complete deletion of the NeoR gene. To determine if the deletion occurred in the germ line, the male offspring was mated to a different heterozygous loxP-NeoR mouse and the genotype of three resulting offspring from the mating determined. One offspring was Cre positive and wild type for the loxP-NeoR allele. One offspring was Cre positive and carried two copies of the deleted allele consistent with inheriting a deleted allele from the male parent and also exhibiting Cre mediated deletion of the loxP-NeoR allele inherited from the female parent. Finally, one mouse that was Cre negative carried a wild type allele and a deleted allele demonstrating germ line transmission of the deleted allele from the father.

Detection of NeoR Excision

In order to detect the extent of loxP-NeoR excision within various tissues, PCR was performed on DNA extracted from various tissues of the Cre positive animal that showed germ line transmission of the deleted allele described above. DNA was prepared from liver, testes, tail, kidney and brain for use in PCR amplifications. All tissues showed deletion of the loxP-NeoR gene as evidenced by the presence of the deleted allele. However, the excision was incomplete in the kidney where a faint intact loxP-NeoR allele was amplified. However, all offspring of this mouse carried only the deleted allele indicating germ line excision was complete.

Cre Expression in Adult Tissues

To assess GFPCre expression in the adult animals, tissues were prepared from a Prion.GFPCre animal and assessed using GFP as a marker. GFP expression was clearly detectable in the adult kidney. In contrast, no other tissue tested (brain, liver and testes) showed GFP expression in adulthood. Therefore, it is unlikely that expression in the adult animal germ line is responsible for deletion of the loxP-flanked NeoR gene during development.

In summary, an inbred 129SvEv transgenic mouse line was generated that allows for reliable and consistent deletion of loxP-flanked sequences in virtually all tissues including the developing germ line.

### Example 3

Behavioral Analysis of Locomotor Activity

Animals were kept on a light-dark schedule of 12 hours with lights on at 6:00 a.m. Behavioral tests were started between 4:30 p.m. and 5:30 p.m. Eleven-to sixteen-week-old mice were assessed in a 15×30 cm cage with four light beams crossing the cage on the long axis. Locomotor activity was measured by computer-automated counting of the number of times a light beam was blocked (San Diego Instruments, San Diego, Calif.). Activity was measured for 3 hours upon introduction of each animal to the chamber on day 1 to measure baseline/novel behavior levels (see FIG. 3), and the animals remained in the chambers for the length of the experiment (3 days) to permit drug response in an acclimated environment. On day 2, animals were injected with saline (i.p),and on day 3 with methylphenidate (7.5 mg/kg or 30.0 mg/kg). Food and water were provided ad libitum, and activity was assessed for 3 hours following injection. Activity scores were subjected to analysis of variance (ANOVA) analysis to compare the groups by genotype and injection status.

The establishment of baseline activity levels shown in FIG. 3 indicated that mice that were either heterozygous or homozygous for the TR mutation demonstrated an increase activity as compared to homozygous wild-type mice. Further, both the heterozygous and homozygous recombinant inbred mice showed decreased levels of activity in response to methylphenidate administration, as compared to an increase in activity for the wild-type mice treated with methylphenidate.

These results indicate that inbred recombinant transgenic mice comprising a mutant TR gene are excellent models for ADHD/ADD. This conclusion is supported by the increased levels of activity demonstrated by the recombinant mice in the absence of treatment. This conclusion is further supported by the observation that the recombinant inbred mice, unlike the wild-type animals, responded to treatment with methylphenidate, an accepted pharmaceutical agent for the treatment of ADHD/ADD. In view of these results it is clear that the recombinant inbred transgenic non-human animals of the disclosed invention will serve as an excellent model for ADHD/ADD.

### Example 4

Behavioral Analysis of Environment Activity

Animals were kept on a light-dark schedule of 12 hours with lights on at 6:00 a.m. Behavioral tests were performed between 8:00 and 17:00. Eighteen to twenty week-old mice were assessed in an ENV-510 Med-associates (http://www.medassociates.com) Open-field Test Environment with a grid of 16×16 IR beams used to continuously monitor location and movement of the animals. Activity was measured for 3 hours upon introduction of each animal to the chamber, and the 3-hour sum of the distance in centimeters was averaged for each genotype. These activity scores were subjected to ANOVA analysis to compare the groups by genotype.

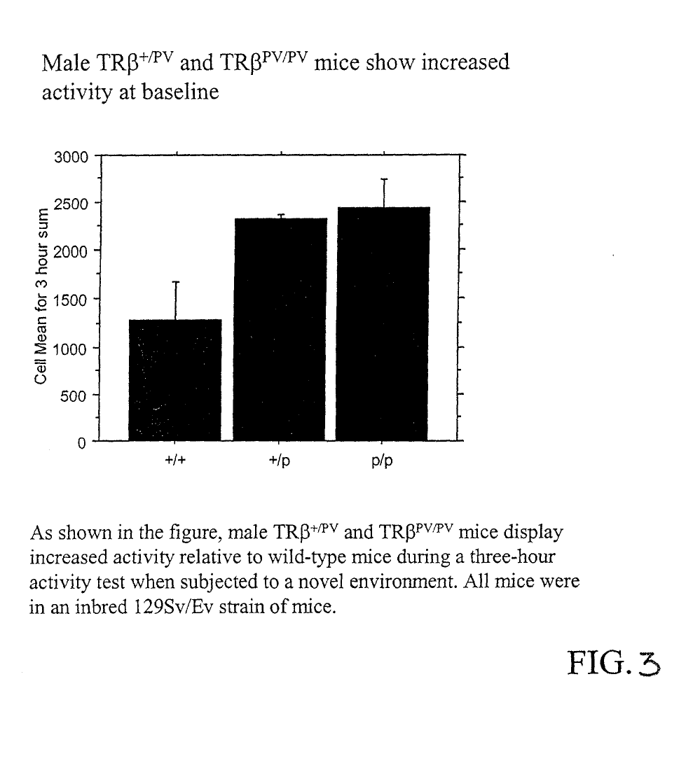

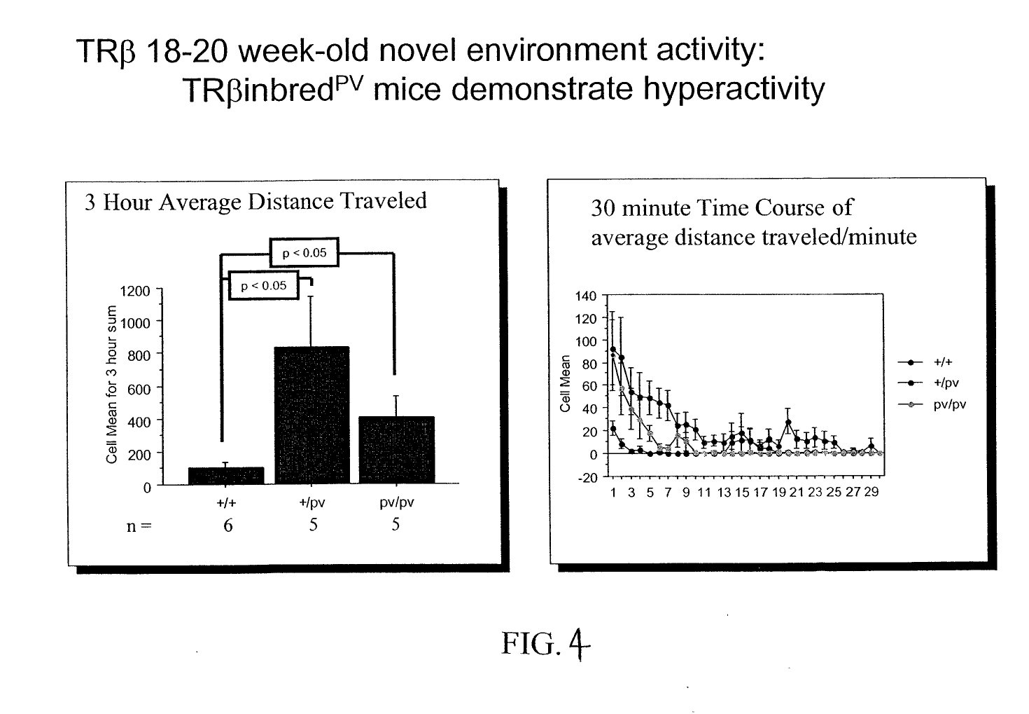

As shown in FIG. 4A, there was a statistically significant variance between the distance traveled by wild-type (+/+) mice, heterozygous (+/PV) mice and homozygous (PV/PV) mice. The TRbinbred+/pv and TRbinbredpv/pv mice were found to demonstrate significantly (p-value<0.05) more activity as measured in distance traveled than wild-type control mice. The cell mean traveled in three hours by wild-type mice was only 100, whereas the heterozygous mice traveled over 800 cells. The homozygous mice were found to have traveled about 400 cells, which is approximately one-half the distance traveled by the heterozygous mice. Accordingly, this demonstrates that mice which are heterozygous or homozygous for the PV mutation are much more active than the wild-type mice.

### Example 5

Determination of Dopamine Release

A panel of compositions of interest are tested for their ability to produce dopamine release from striata harvested from transgenic mice produced using that methods described herein. Methylphenidate hydrochloride is used a positive control against which the release of dopamine from the transgenic tissue is compared.

Striata from four transgenic mice expressing a TR-beta thyroid hormone receptor containing the PV mutation are homogenized in 2 ml of 0.32M sucrose buffered with 5 mM HEPES (pH 7.5), using a glass-Teflon tissue grinder. The homogenate is then diluted to 5 ml with additional homogenization solution and centrifuged at 1,000×g for 10 minutes. This procedure is repeated on the new pellet and the resulting supernatant is centrifuged at 12,000×g for 20 minutes. A 3 layer discontinuous Percoll gradient consisting of 16 percent, 10 percent and 7.5 percent Percoll in HEPES-buffered sucrose is made with the final pellet dispersed in the top layer.

After centrifugation at 15,000×g for 20 minutes, the synaptosomes are recovered above the 16 percent layer with a Pasteur pipette, diluted with 8 ml of perfusion buffer (128 mM NaCl, 2.4 mM KCl, 3.2 mM CaCl₂, 1.2 mM KH₂ PO₄, 1.2 mM MgSO₄, 25 mM HEPES pH 7.4, 10 mM dextrose, 1 mM ascorbate, 0.01 mM pargyline), and centrifuged at 15,000×g for 20 minutes.

The new pellet is collected and re-suspended in perfusion buffer. The synaptosome suspension is incubated for 10 minutes at 37° C. Tritium labeled dopamine (Amersham, 40-60 Ci/mmol) is added to the suspension to give a final concentration of 0.1 μM, and the suspension is incubated for another 5 minutes.

Using this method, 30 to 90 percent of the dopamine is taken up into the synaptosomes, as determined by scintillation counting following filtration through glass fiber filters soaked with 0.5 percent polyethyleneimine. A continuous perfusion system is used to monitor release following exposure to each compound of interest. Synaptosomes are loaded onto glass fiber filters (Gelman type A/E). Perfusion buffer is dripped onto the filters (0.2-0.3 ml/minutes) and pulled through the filters with a peristaltic pump. Synaptosomes are washed with perfusion buffer for a minimum of 20 minutes before addition of the compound of interest. After the addition of 0.2 ml of a solution containing various concentrations of ligand, the perfusate is collected into scintillation vials at 1 minute intervals and the dopamine released is quantified by scintillation counting. Peaks of radioactivity released above background are summed and the average basal release during that time is subtracted from the total. Release is expressed as a percentage of release obtained with an equal concentration of methylphenidate hydrochloride.

### Conclusion

Although the invention has been described with reference to embodiments and examples, it should be understood that various modifications can be made without departing from the spirit of the invention. Accordingly, the invention is limited only by the following claims.

What is claimed is:

1. An inbred non-human transgenic animal comprising a dominant-negative mutated nuclear hormone receptor, wherein said transgenic animal has an Attention Deficit Disorder/Attention Deficit Hyperactivity Disorder (ADD/ADHD) phenotype.

2. The inbred non-human transgenic animal of claim 1, wherein the mutated nuclear hormone receptor is thyroid hormone receptor-beta.

3. The inbred non-human transgenic animal of claim 2 wherein the non-human transgenic animal is a mouse.

4. The inbred non-human transgenic animal of claim 2, wherein the non-human transgenic animal is an inbred mouse of line 129 SvEV.

5. The inbred non-human transgenic animal of claim 2, wherein the mutated thyroid hormone receptor-beta is the PV mutant.

6. An inbred non-human transgenic animal comprising a mutated homodimer or heterodimer partner of a nuclear hormone receptor, wherein said transgenic animal has an Attention Deficit Disorder/Attention Deficit Hyperactivity Disorder (ADD/ADHD) phenotype.

7. The inbred non-human transgenic animal of claim 6, wherein the thyroid hormone receptor is thyroid hormone receptor-beta.

8. The inbred non-human transgenic animal of claim 6, wherein the homodimer or heterodimer partner is selected from the group consisting of: thyroid hormone receptor-alpha (TRa), thyroid hormone receptor-beta (TRb), retinoic acid receptor-alpha (RARa), retinoic acid receptor-beta (RARb), retinoic acid receptor-gamma (RARg), retinoid X receptor-alpha (RXRa), retinoid X receptor-beta (RXRb), retinoid X receptor-gamma (RXRg), estrogen receptor (ER), progesterone receptor (PR), androgen receptor (AR), glucocorticoid receptor (GR), mineralocorticoid receptor (MR), and vitamin D receptor (VDR)

9. A method for producing an inbred non-human transgenic animal strain having an Attention Deficit Disorder/Attention Deficit Hyperactivity Disorder (ADD/ADHD) phenotype, comprising:
providing a targeting vector comprising a dominant negative mutated nuclear hormone receptor polynucleotide sequence;
introducing said targeting vector into a plurality of animal embryos to produce a plurality of transgenic animals; and
selecting a transgenic animal that exhibits the ADD/ADHD phenotype.

10. The method of claim 9, comprising crossbreeding said transgenic animal with a homozygous Ella-Cre transgenic animal of the same strain.

11. The method of claim 9, wherein said transgenic animal comprises a recombinant nuclear hormone receptor having a lower affinity for its cognate ligand as compared to a corresponding wild-type nuclear hormone receptor.

12. The inbred non-human transgenic animal of claim 9, wherein the mutated nuclear hormone receptor is thyroid hormone receptor-beta.

13. The inbred non-human transgenic animal of claim 9, wherein the mutated thyroid hormone receptor-beta is the PV mutant.
